(19) United States
(12) Patent Application Publication
(10) Pub. No.: US 2011/0092862 A1
(43) Pub. Date: Apr. 21, 2011

(54) COUPLING MATERIAL

(75) Inventor: Robin Chivers, York (GB)

(73) Assignee: SMITH & NEPHEW PLC, London (GB)

(21) Appl. No.: 12/977,689

(22) Filed: Dec. 23, 2010

Related U.S. Application Data
(62) Division of application No. 11/912,381, filed on Jun. 30, 2008.

(30) Foreign Application Priority Data
Apr. 23, 2005 (GB) 0508250.8

(51) Int. Cl. A61N 7/00

(52) U.S. Cl. 601/2

(57) ABSTRACT

A solid coupling material for conducting ultrasound from an ultrasound source to a surface, wherein the material maximises contact with the surface and the ultrasound source such that there are minimal or no air gaps in the interface between the surface and the material and in the interface between the ultrasound source and the material.

### CROSS-REFERENCE TO RELATED APPLICATIONS

This application is a divisional of U.S. patent application Ser. No. 11/912,381, which is a National Stage of International Application No. PCT/GB2006/0013778, filed Apr. 13, 2006, which claims the benefit of Priority Document No. 0508250.8, filed Apr. 23, 2005. The disclosure of each application is incorporated by reference in its entirety.

### BACKGROUND

The present invention relates to a solid coupling material for conducting ultrasound from an ultrasound source to a surface, particularly a skin surface of a patient. This invention also relates to an ultrasound device incorporating such an acoustic coupling material. This invention also relates to a method of coupling an ultrasound source with a surface. This invention also relates to a method of treating a patient with ultrasound.

The use of ultrasound in medical applications is well documented. The use of ultrasound for the healing of bone fractures is also well known (see for example U.S. Pat. No. 4,530,360 and U.S. Pat. No. 5,520,612).

It is known to couple ultrasound transducers to a patient's skin with “coupling gels”. These fluid coupling gels tend to be water-based materials. These permit the removal of any air from the interface between the transducer and the skin, so that ultrasound may be freely transmitted. In most ultrasound applications, the fluid gel is advantageous as the transducer has to be moved on the skin surface. The fluid nature of the gel means that its application can cause a significant mess.

Solid gels are known, which are primarily used for cushioning. These usually consist of a thermoplastic elastomer with a mineral oil incorporated into it. These materials can ^{“}weep” very small quantities of mineral oil upon the application of high pressure. These materials therefore have an intrinsically dry surface.

The present invention aims to provide a solid coupling material that is clean (does not cause a mess when applied to the surface) and which permits an ultrasound transducer to be well coupled to a surface so that ultrasound can be conducted to the surface unhindered by the interface. For example, the ultrasound transducer can be well coupled to the skin of a patient so that the therapeutic ultrasound signal can enter the body unhindered by the interface.

### SUMMARY

According to a first aspect of the present invention, there is provided a solid coupling material for conducting ultrasound from an ultrasound source to a surface, wherein the material maximises contact with the surface and the ultrasound source such that there are minimal or no air gaps in the interface between the surface and the material and in the interface between the ultrasound source and the material.

The solid nature of the coupling material has the advantage that it is clean compared to known fluid gels. By maximising the contact with the surface such that there are minimal or no air gaps in the interface between the surface and the material, the ultrasound signal is transmitted to the surface unhindered.

According to an embodiment of the invention, the surface is a skin surface, for example of a patient.

According to an embodiment of the invention, the material is deformable so that it can accommodate the shape of the surface and thereby maximise contact with the surface.

According to an embodiment of the invention, the material comprises a fluid.

Preferably, the fluid is exuded by the material to form a fluid layer that maximises contact with the surface.

Preferably, the fluid is sufficiently mobile so that a film or layer will readily wet the surface upon the application of light/minimal pressure to the material. The resulting fluid film or layer will ensure a good path for the ultrasound when the material is pressed onto the surface and onto the transducer.

The fluid may be made sufficiently mobile by reducing the fluid's molecular weight. The fluid may be made sufficiently mobile by selecting a suitable composition.

The fluid may comprise water. The fluid may comprise an aqueous solution.

The fluid may comprise at least one organic liquid. The fluid may comprise at least one hydrocarbon liquid. The fluid may comprise at least one oil. The fluid may comprise at least one alkane. The at least one alkane may be a C₆-C₁₂ alkane, preferably a C₈-C₁₀ alkane.

In those embodiments of the invention in which the material comprises a fluid, preferably the material has an intrinsically wet surface.

The fluid may provide about 5-95% of the total weight of the material. The fluid may provide about 10-95% of the total weight of the material. The fluid may provide about 25-95% of the total weight of the material. The fluid may provide about 45-95% of the total weight of the material. The fluid may provide about 65-95% of the total weight of the material. Preferably, the fluid provides about 75-95% of the total weight of the material. More preferably, the fluid provides about 80-90% of the total weight of the material.

The fluid may be uniformly distributed throughout the material. The fluid may be predominantly disposed at or near the surface of the material.

The fluid may have beneficial/therapeutic properties, for example moisturising properties. The fluid may be absorbed by a skin surface. This would also help to prevent mess.

The fluid may be present in the material naturally or it may be incorporated into the material by artificial means. The manufacture of the material may be by any known means.

The actual fluid exuded may be a variety of particular fluids, solutions and/or combinations of fluids/solutions. Aptly the fluid may be clear. Aptly the fluid will not have an unpleasant odour.

The exudation of fluid by the material may be caused by the application of ultrasound. The exudation of fluid by the material may be caused by the application of pressure, for example by pressing the material against the patient's skin.

The material may in some embodiments of the present invention be attached to the transducer. In other embodiments it may not necessarily be attached to the transducer.

In some embodiments of the present invention the material may have an indication means to indicate when it is unable to exude sufficient fluid to sufficiently act as an ultrasonic conductive material.

The indicative means may be a colour change. For example, in particular embodiments of the present invention a colour change from one colour to another colour would indicate that the material did not have sufficient fluid to exude to sufficiently act as an ultrasonic conductive material.

Preferably, the material is substantially homogeneous. Thus, the material may be free, or substantially free, of voids. The material may be free, or substantially free, of filler particles. Homogeneous materials are more transparent to ultrasound, minimising the loss of energy for ultrasound travelling through them.

The material may comprise a solid gel. Preferably, the material comprises a polymer. Preferably, the polymer is an elastomer. Preferably, the polymer has a low glass transition temperature (T_{g}). Preferably, the polymer is a rubber. The rubber may be a synthetic or natural rubber. Preferably, the rubber is synthetic in order to avoid possible detrimental effects such as skin sensitisation.

Preferably, the polymer has a low cross-link density. In those embodiments of the invention in which the material comprises a fluid, a low cross-link density for the polymer can result in increased mobility for the fluid.

The material may comprise a copolymer of styrene, ethylene and butadiene.

Preferably, the material comprises a highly extensible elastomer and a high level of oil. Preferably, the material comprises an oil covered surface.

In those embodiments of the invention in which the material is deformable, the material may be a soft elastomer or an aqueous equivalent, e.g. a hydrogel.

Soft elastomers are particularly suitable in this regard. Soft elastomers are typically rubbers with a low glass transition temperature (T_{g}). Usually, the rubbers contain some oil to reduce the T_{g}. Any rubber which is compatible with suitable oils may be used. The rubber should contain minimal, preferably no, filler or voids (bubbles). In these embodiments of the invention, oil may be required in order to make the rubber soft enough, but does not necessarily need to ‘wet’ a surface if the rubber is soft enough to make sufficient contact with the surface.

The material may have “tack” such that the forces of adhesion between the material and a surface ensure that contact is maximised such that there are minimal or no air gaps in the interface between the surface and the material. Soft elastomers are particularly suitable in this regard as they have inherent “tack”.

Embodiments of the present invention therefore relate to syneresing materials including gels, rubbers, polymers etc. and applications thereof

Syneresing materials are materials that leak out fluids such as water or oil from the materials structure. This is also commonly known as “blooming”. Hence syneresing materials are in accordance with the present invention.

Embodiments of the present invention use syneresing materials to act as an ultrasonic conductive gel on or with an ultrasonic (wave) treatment device or transducer.

In order for an ultrasonic treatment session to be beneficial to a patient, at least a portion of the ultrasound wave must penetrate the body and reach the injury to accelerate the healing process. In order to minimize excessive attenuation of the ultrasound waves produced by the transducer, an ultrasonic wave coupling material, e.g. a conductive gel, is used between the surface of the skin and the transducer head. If an inadequate amount of gel is used or it is improperly applied on the patient, the treatment session will not be as effective as it should be.

Ideally the syneresing material of the present invention will exude an oil that will act as an efficient conductive gel to the ultrasonic transducer. It may exude an aqueous solution, mixture of oils or other.

This oil (or aqueous solution, mixture of oils or other) may be present in the syneresing material naturally or it may be incorporated into the material by artificial means in order to give a syneresing material.

The manufacture of the syneresing material may be by any known means.

The actual oil exuded may be a variety of particular oils, solutions and /or combinations of oils/solutions. Aptly the oil may be clear. Aptly the oil will not have an unpleasant smell.

The oil (or exuding liquid etc.) may also have beneficial properties to the skin e.g. like moisturising oil. The oil may actually be absorbed by the skin. This would prevent mess.

The material exudation may be started, in some embodiments, by the ultrasound, or in other embodiments from pressure such as pressing against the patient's skin.

The syneresing material may in some embodiments of the present invention be attached to the transducer. In other embodiments it may not necessarily be attached to the transducer.

In some embodiments of the present invention the syneresing material may have an indication means when it is unable to exude sufficient gel to sufficiently act as an ultrasonic conductive gel.

The indicative means may be a colour change in particular embodiments of the present invention on which a colour change from one colour to another colour would indicate that the gel did not have sufficient oil to exude to sufficiently act as an ultrasonic conductive gel.

According to a second aspect of the present invention, there is provided an ultrasound device, comprising a coupling material according to the first aspect of the present invention and an ultrasound source coupled to the coupling material.

The coupling material according to the first aspect of the present invention may have a means for connecting itself to the ultrasound source.

The ultrasound device may comprise a coupling material according to the first aspect of the invention attached to an ultrasound transducer, wherein the contact between the ultrasound transducer and the material is maximised such that there are minimal or no air gaps in the interface between the ultrasound transducer and the material.

According to a third aspect of the present invention, there is provided a method of coupling an ultrasound source with a surface, comprising the steps of providing a coupling material according to the first aspect of the present invention, providing an ultrasound source, coupling the coupling material with the ultrasound source, and coupling the coupling material with the surface.

According to a fourth aspect of the present invention, there is provided a method of treating a patient with ultrasound, comprising the steps of providing a coupling material according to the first aspect of the present invention, providing an ultrasound source, coupling the coupling material with the ultrasound source; coupling the coupling material with the skin of the patient, and activating the ultrasound source so that ultrasound is conducted into the patient.

According to a fifth aspect of the present invention, there is provided a material comprising a polymer and a low molecular weight organic liquid, wherein the organic liquid provides about 5-95% of the total weight of the material.

The organic liquid may provide about 10-95% of the total weight of the material. The organic liquid may provide about 25-95% of the total weight of the material. The organic liquid may provide about 45-95% of the total weight of the material. The organic liquid may provide about 65-95% of the total weight of the material. Preferably, the organic liquid provides about 75-95% of the total weight of the material. More preferably, the organic liquid provides about 80-90% of the total weight of the material.

The polymer may be a butadiene rubber. Preferably, the butadiene rubber is cis-butadiene rubber. Preferably, the cis-butadiene rubber is peroxide cured. The organic liquid may be an oil. Preferably, the oil is a paraffin oil. Preferably, the paraffin oil is a light paraffin oil.

The paraffin oil may be a C₆-C₁₂ paraffin. Preferably, the paraffin oil is a C₈-C₁₀ paraffin.

The material may comprise 1-9 parts oil and 9-1 parts rubber, as appropriate. Preferably, the material comprises 3-9 parts oil and 7-1 parts rubber, as appropriate. More preferably, the material comprises 3-7 parts oil and 7-3 parts rubber, as appropriate.

### BRIEF DESCRIPTION OF THE DRAWINGS

Reference will now be made, by way of example, to the accompanying figures, in which:

FIG. 1 shows a coupling material in accordance with an embodiment of the present invention;

FIG. 2 shows a coupling material in accordance with another embodiment of the present invention;

FIG. 3 shows a coupling material in accordance with an embodiment of the present invention coupled to an ultrasound transducer and a surface; and

FIG. 4 shows a coupling material in accordance with another embodiment of the present invention coupled to an ultrasound transducer and a surface.

### DETAILED DESCRIPTION OF THE EMBODIMENTS

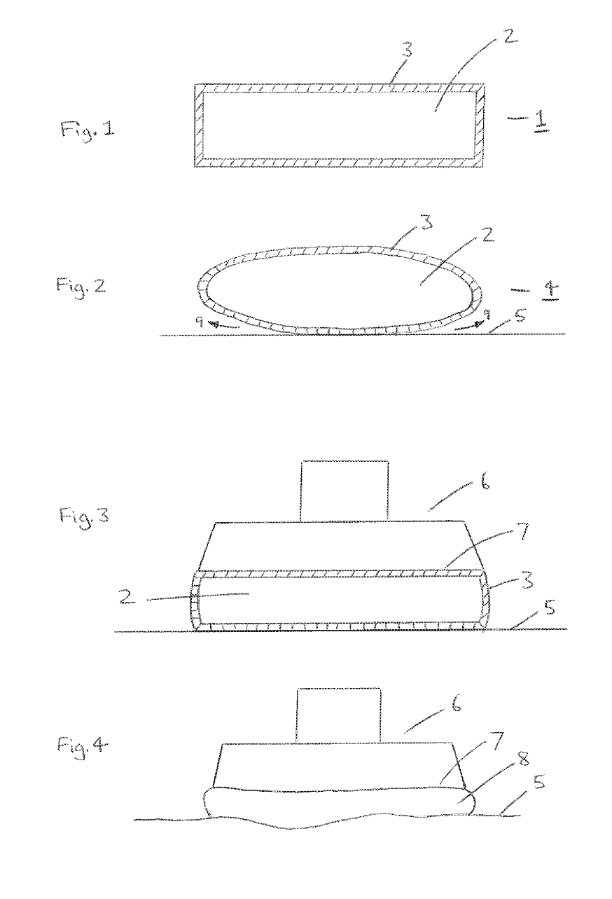

FIGS. 1 and 2 show solid coupling materials (1,4) in accordance with the present invention having alternative shapes. In the embodiment shown in FIG. 1, the coupling material (1) is substantially rectangular in cross-section. In the embodiment shown in FIG. 2, the coupling material (4) is substantially convex in cross-section. Both coupling materials (1,4) comprise a solid body (2) having a fluid layer (3) on their outer surfaces.

The embodiment of FIG. 2 has the advantage that when it is positioned in contact with a surface (5), air is displaced as shown by arrows (9). This further minimises/prevents the formation of air gaps/pockets, which are detrimental to the performance of ultrasound devices in accordance with the present invention.

FIG. 3 shows a coupling material (1,4) coupled to an ultrasound transducer (6) and a surface (5), for example the surface of a patient's skin. As shown, the fluid layer (3) maximises contact with the surface (7) of the transducer (6) and with the surface (5), such that there are minimal or no air gaps in the interfaces between the surfaces (5,7) and the material (1,4).

FIG. 4 shows a solid coupling material (8) which is deformable so that it can accommodate the shape of surface (5) and surface (7) of the transducer (6). As a result, the contact with the surface (7) of the transducer (6) and with the surface (5) is maximised such that there are minimal or no air gaps in the interfaces between the surfaces (5,7) and the material (8). The material (8) may be a soft elastomer or an aqueous equivalent, e.g. a hydrogel.

Soft elastomers are particularly suitable in this regard. Soft elastomers are typically rubbers with a low glass transition temperature (T_{g}). Usually, the rubbers contain some oil to reduce the T_{g}. Any rubber which is compatible with suitable oils may be used. The rubber should contain minimal, preferably no, filler or voids (bubbles). In this embodiment of the invention, oil may be required in order to make the rubber soft enough, but does not necessarily need to ‘wet’ the surfaces (5,7) if the rubber is soft enough to make sufficient contact with the surfaces (5,7).

The material (8) may have “tack” such that the forces of adhesion between the material (8) and the surfaces (5,7) ensure that contact is maximised such that there are minimal or no air gaps in the interfaces between the surfaces (5,7) and the material (8). Soft elastomers are particularly suitable in this regard as they have inherent “tack”.

### EXAMPLES

A preferred solid coupling material comprises: high cis-butadiene rubber (supplied by National Petrochemical Co., Iran; equivalent to Enichem's Europrene Cis); Dicumyl Peroxide (crosslinker); and Strukthene 410 (low viscosity naphthenic oil, supplied by Safic Alcan).

This material may be prepared by the following steps: chop the rubber into small pieces; add the oil and stir with a z-blade mixer until the mixture is homogeneous (no heating is involved); add the crosslinker; and cast and cover while it cures.

The material may also be prepared by swelling the oil into a crosslinked rubber.

### Example 1

The coupling material comprises: 100 pph high cis-butadiene rubber; 1 pph Dicumyl Peroxide; and 900 pph Strukthene 410.

### Example 2

The coupling material comprises: 100 pph high cis-butadiene rubber; 1 pph

Dicumyl Peroxide; and 400 pph Strukthene 410.

### Example 3

The coupling material comprises: 1 part Kraton G 1650/1652 (copolymer based on styrene/ethylene/butadiene) with 9 parts light paraffin oil (C_{8-C}₁₀ alkanes).

### Comparative Example 4

Commercially available medical ultrasound fluid coupling gel (LithoClear).

### Comparative Example 5

Commercially available solid cushioning gel consisting of a rubber with mineral oil incorporated into it and a kaolin dusted surface.

The following table provides ultrasound transmission data for the above materials.

| | Proportion of power |
|---|---|
| | transmitted from |
| Example | transducer in contact. |
| 1 | 92% |
| 2 | 98% |
| 3 | 92% |
| 4 | 95% |
| 5 | 8% |

As can be seen from the table, materials in accordance with the invention (examples 1-3) transmit a high proportion of the ultrasound, having transmission characteristics similar to, if not better than, commercially available ultrasound coupling gel (example 4). The solid coupling materials of the present invention have the further advantage that they are clean compared to the fluid gels, as described earlier.

Comparative example 5 provides evidence that a known rubber-based material that has an intrinsically dry surface transmits a very low proportion of ultrasound and is therefore not suitable as an ultrasound coupling material.

The solid coupling material may also comprise a polyurethane elastomer containing gycol oil.

### Example 6

An oil loaded polyurethane elastomer was prepared by the following method: Desmodur N3200 (23.4 g) was placed in a reaction vessel and a polyethylene glycol-polypropylene glycol monobutyl ether random copolymer (176.6 g, MW 3900) was added together with bismuth tris neodecanoate/decanoic acid catalyst (0.04 g, Coscat 83). The mixture was heated at 70° C. for 6 hours to form a pre-polymer.

A portion of this pre-polymer (55.0 g) was placed in a reaction vessel and a polyethylene glycol-polypropylene glycol-polyethylene glycol block copolymer (20.0 g) was added together with Coscat 83 (0.004 g) and the mixture stirred at room temperature for 30 seconds. Diethylene glycol dibutyl ether (300 g) was added and the mixture stirred for 10 seconds and poured into a glass dish. The resulting reaction mixture was cured at 70° C. for 12 hours.

Dipropylene glycol dimethyl ether can also be used in place of diethylene glycol dibutyl ether.

### Example 7

An oil loaded polyurethane elastomer was prepared by the following method: Desmodur E305 (16.6 g) was placed in a reaction vessel and Levagel VPKA 8732 (83.09 g) was added together with Coscat 83 catalyst. The reaction mixture was stirred for 1 minute. Diethylene glycol dibutyl ether (300 g) was added and the mixture stirred for 10 seconds and poured into a glass dish. The resulting reaction mixture was cured at 70° C. for 12 hours.

Dipropylene glycol dimethyl ether can also be used in place of diethylene glycol dibutyl ether.

Desmodur 3200 is an isocyanate terminated polyether pre-polymer on a base of hexamethylene diisocyanate (2 functional).

Levagel VPKA 8732 is a polyether polyhydric alcohol (4 functional).

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. An ultrasound system comprising:
an ultrasound source; and
a solid coupling material for conducting ultrasound from the ultrasound source to a surface, wherein the material contacts the surface and the ultrasound source,
wherein the material comprises a liquid having an oil component, and is configured such that the liquid exudes from the material to form a first liquid layer between the material and the surface and to form a second liquid layer between the material and the ultrasound source, the first liquid layer mitigating against formation of one or more gas pockets between the surface and the material and the second liquid layer mitigating against a formation of one or more gas pockets between the ultrasound source and the material.

2. The ultrasound system according to claim 1, wherein the material is deformable so that it can accommodate the shape of the surface and the ultrasound source.

3. The ultrasound system according to claim 1, wherein the oil component comprises at least one alkane.

4. The ultrasound system according to claim 3, wherein the at least one alkane is a C₆-C₁₂ alkane.

5. The ultrasound system according to claim 4, wherein the at least one alkane includes a C₈-C₁₀ alkane.

6. The ultrasound system according to claim 1, wherein the material comprises a polymer.

7. The ultrasound system according to claim 6, wherein the polymer is an elastomer.

8. The ultrasound system according to claim 7, wherein the polymer is a rubber.

9. The ultrasound system according to claim 7, wherein the polymer has a low T_{g.}

10. The ultrasound system according to claim 6, wherein the polymer has a low cross-link density.

11. The ultrasound system according to claim 6, wherein the material comprises a copolymer of styrene, ethylene and butadiene.

12. The ultrasound system according to claim 6, wherein the material is substantially homogeneous.

13. The ultrasound system according to claim 6, wherein the material comprises a highly extensible elastomer and a high level of oil.

14. The ultrasound system according to claim 12, comprising an oil-covered surface.

15. The ultrasound system according to claim 1, wherein the fluid provides about 5-95% of the total weight of the material.

16. The ultrasound system according to claim 1, wherein the fluid provides about 80-90% of the total weight of the material.

17. The ultrasound system according to claim 1, wherein the material is tacky and adheres to the surface and to the ultrasound source.

18. An ultrasound applicator kit comprising:
an ultrasound source; and
a coupling material comprising a polymer and a low molecular weight organic liquid,
wherein the organic liquid provides about 5-95% of the material, the material being configured such that the organic liquid exudes from the polymer to form a liquid layer on an external surface of the material.

19. The ultrasound applicator kit according to claim 18, wherein the organic liquid provides about 80-90% of the total weight of the material.

20. The ultrasound applicator kit according to claim 18, wherein the polymer is a butadiene rubber and the organic liquid is a light paraffin oil.

21. The ultrasound applicator kit according to claim 20, wherein the polymer is a cis-butadiene rubber.

22. The ultrasound applicator kit according to claim 18, wherein the polymer is polyurethane and the organic liquid is a glycol oil.

23. The ultrasound applicator kit according to claim 18, wherein the material is tacky.
